(19) United States
(12) Patent Application Publication
(10) Pub. No.: US 2026/0285790 A1
(43) Pub. Date: Sep. 24, 2026

(54) METHOD FOR PURIFICATION OF ETHANOL USING EXTRACTIVE DISTILLATION

(71) Applicant: Air Company Holdings, Inc., Brooklyn, NY (US)

(72) Inventors: Konstantina Kritikos, Brooklyn, NY (US); Yinxian Wang, Brooklyn, NY (US); Stafford W. Sheehan, Tiverton, RI (US); Chi Chen, Cliffside Park, NJ (US); Lubo Zhou, Deer Park, IL (US)

(21) Appl. No.: 19/473,880

(22) PCT Filed: Apr. 9, 2024
(86) PCT No.: PCT/US24/23661

§ 371 Date: Oct. 9, 2025

Related U.S. Application Data
(60) Provisional application No. 63/495,317, filed on Apr. 11, 2023.

(51) Int. Cl. C07C 29/84

(52) U.S. Cl. CPC C07C 29/84

(57) ABSTRACT

Provided herein are methods and systems for the purification of food-grade ethanol using extractive distillation with propylene glycol as the extraction solvent. The systems and processes include one or more separators and distillation columns, and optionally provide a propylene glycol recycle stream.

### CROSS REFERENCE TO RELATED APPLICATION

This application claims the benefit of priority to U.S. Provisional Patent Application 63/495,317, filed Apr. 11, 2023; the entire contents of which are incorporated herein by reference.

### BACKGROUND

Separation of impurities from ethanol suitable for use in the beverage industry provides a unique set of challenges due to the need for extremely high purity products and the need for removal of all potentially toxic byproducts from the manufacturing process. Extractive distillation provides an effective way to produce high purity ethanol; however, current solvents used in such processes are unsuitable for food-grade applications due to their toxicity. Such purification processes are made more difficult due to the formation of azeotropes when purifying ethanol in the presence of water and other alcohols. Improved methods for purifying ethanol are needed.

### SUMMARY OF THE INVENTION

Provided herein are methods for the purification of food-grade ethanol. A method includes: (1) combining an alcohol feed comprising ethanol and propanol with a propylene glycol feed comprising propylene glycol to produce a purification feed; (2) distilling the purification feed at an extractive distillation temperature and an extractive distillation pressure to produce an ethanol product mixture comprising ethanol and a propylene glycol mixture comprising propylene glycol and propanol; and (3) separating the propylene glycol from the propanol in the propylene glycol mixture at a separation temperature and a separation pressure to produce a propanol product mixture comprising propanol and a propylene glycol product mixture comprising propylene glycol. The method may comprise combining the propylene glycol product mixture with the propylene glycol feed. The method may comprise distilling a crude alcohol feed comprising methanol, ethanol, and propanol at a crude alcohol distillation temperature and a crude alcohol distillation pressure to produce the alcohol feed and a methanol product mixture comprising methanol. The method may comprise separating a hydrated alcohol feed comprising water, methanol, ethanol, and propanol at a hydrated alcohol separation temperature and hydrated alcohol separation pressure to produce the crude alcohol feed and a waste water mixture comprising water.

Also provided herein are systems for the purification of food-grade ethanol. That system may include: an alcohol feed; a propylene glycol feed; an extractive separator comprising an alcohol feed inlet, a propylene glycol feed inlet, an ethanol outlet, and a propylene glycol mixture outlet. The alcohol feed may be coupled to the alcohol feed inlet, and the propylene glycol feed may be coupled to the propylene glycol feed inlet. A propylene glycol separator may comprise a propylene glycol mixture inlet, a propanol outlet, and a propylene glycol outlet. The propylene glycol mixture inlet may be coupled to the propylene glycol mixture outlet from the extractive separator.

The propylene glycol outlet from the propylene glycol separator may be coupled to the propylene glycol feed. The system may comprise a crude alcohol feed, and/or a methanol separator comprising a crude alcohol feed inlet, a methanol outlet, and an alcohol feed outlet. The crude alcohol feed may be coupled to the crude alcohol feed inlet, and the alcohol feed outlet may be coupled to the alcohol feed. The system may comprise a hydrated alcohol feed; and, optionally, a water separator comprising a hydrated alcohol feed inlet, a water outlet, and a crude alcohol feed outlet. The hydrated alcohol feed may be coupled to the hydrated alcohol feed inlet, and the crude alcohol feed outlet is coupled to the crude alcohol feed.

### BRIEF DESCRIPTION OF THE DRAWINGS

FIG. 1 is an example of a process schematic for the purification of food-grade ethanol from a hydrated alcohol feed using a number of separators and extractive distillation.

FIG. 2 is an example of a process schematic for the extractive distillation of propanol impurities from an alcohol feed using extractive distillation with propylene glycol as the extraction solvent.

### DETAILED DESCRIPTION OF THE INVENTION

Provided herein are methods and systems for the purification of food-grade ethanol using extractive distillation with propylene glycol as the extraction solvent.

### Methods of Extractive Distillation

Methods for the purification of food-grade ethanol are disclosed herein. The method includes: combining (a) an alcohol feed comprising ethanol and propanol with (b) a propylene glycol feed comprising propylene glycol to produce a purification feed; distilling the purification feed at an extractive distillation temperature and an extractive distillation pressure to produce (a) an ethanol product mixture comprising ethanol and (b) a propylene glycol mixture comprising propylene glycol and propanol; and separating the propylene glycol from the propanol in the propylene glycol mixture at a separation temperature and a separation pressure to produce (a) a propanol product mixture comprising propanol and (b) a propylene glycol product mixture comprising propylene glycol.

The alcohol feed may comprise greater than about 85% (v/v) ethanol, greater than about 90% (v/v) ethanol, greater than about 93% (v/v) ethanol, greater than about 95% (v/v) ethanol, or greater than about 98% (v/v) ethanol. The alcohol feed may comprise about 85% (v/v) to less than about 100% (v/v) ethanol. The alcohol feed may comprise about 90% (v/v) to less than about 100% (v/v) ethanol. The alcohol feed may comprise about 88% (v/v) to about 99% (v/v) ethanol. The alcohol feed may comprise about 90% (v/v) to about 99% (v/v) ethanol. The alcohol feed may comprise about 92% (v/v) to about 99% (v/v) ethanol. The alcohol feed may comprise about 95% (v/v) to about 99% (v/v) ethanol. The alcohol feed may comprise about 90% (v/v), about 92% (v/v), about 95% (v/v), about 97% (v/v), about 98% (v/v), or about 99% (v/v) ethanol.

The molar ratio of the alcohol feed to the propylene glycol feed in the purification feed may be about 1:0.5 to about 1:6, or about 1:1 to about 1:3. The molar ratio of the alcohol feed to the propylene glycol feed may be about 1:0.75. The molar ratio of the alcohol feed to the propylene glycol feed may be about 1:1. The molar ratio of the alcohol feed to the propylene glycol feed may be about 1:2. The molar ratio of the alcohol feed to the propylene glycol feed may be about 1:3. The molar ratio of the alcohol feed to the propylene glycol feed may be about 1:4. The molar ratio of the alcohol feed to the propylene glycol feed may be about 1:5. In certain embodiments, these ratios represent the ratios of the ethanol plus the propanol in the alcohol feed to the propylene glycol in the propylene glycol feed.

The propanol may comprise i-propanol and/or n-propanol. The alcohol feed may comprise less than about 20% (v/v), less than about 15% (v/v), less than about 12% (v/v), less than about 10% (v/v), or less than about 9% (v/v) propanol. The alcohol feed may comprise about 1% (v/v) to about 20% (v/v) propanol, about 5% (v/v) to about 20% (v/v) propanol, about 5% (v/v) to about 15% (v/v) propanol, about 5% (v/v) to about 12% (v/v) propanol, or about 8% (v/v) to about 10% (v/v) propanol. The propanol may comprise i-propanol, n-propanol or a combination of both i-propanol and n-propanol.

The alcohol feed may comprise less than about 15% (v/v) n-propanol. The alcohol feed may comprise less than about 10% (v/v) n-propanol. The alcohol feed may comprise less than about 5% (v/v) n-propanol. The alcohol feed may comprise less than about 1% (v/v) n-propanol. The alcohol feed may comprise about 0.1% (v/v) to about 15% (v/v) n-propanol, about 1% (v/v) to about 10% (v/v) n-propanol, or about 5% (v/v) to about 10% (v/v) n-propanol. The alcohol feed may comprise about 10% (v/v) n-propanol. The alcohol feed may comprise about 5% (v/v) n-propanol. The alcohol feed may comprise about 1% (v/v) n-propanol.

The alcohol feed may comprise less than about 15% (v/v) i-propanol. The alcohol feed may comprise less than about 10% (v/v) i-propanol. The alcohol feed may comprise less than about 5% (v/v) i-propanol. The alcohol feed may comprise less than about 1% (v/v) i-propanol. The alcohol feed may comprise about 0.1% (v/v) to about 15% (v/v) i-propanol, about 0.5% (v/v) to about 10% (v/v) i-propanol or about 0.1% (v/v) to about 5% (v/v) i-propanol. The alcohol feed may comprise about 10% (v/v) i-propanol. The alcohol feed may comprise about 5% (v/v) i-propanol. The alcohol feed may comprise about 1% (v/v) i-propanol.

The alcohol feed may comprise greater than 80% (v/v) ethanol, less than about 15% (v/v) i-propanol, and less than about 15% (v/v) n-propanol. The alcohol feed may comprise greater than 80% (v/v) ethanol, less than about 10% (v/v) i-propanol, and less than about 10% (v/v) n-propanol. The alcohol feed may comprise greater than 85% (v/v) ethanol, less than about 5% (v/v) i-propanol, and less than about 15% (v/v) n-propanol. The alcohol feed may comprise greater than 90% (v/v) ethanol, less than about 1% (v/v) i-propanol, and less than about 10% (v/v) n-propanol.

The alcohol feed may comprise additional impurities, such as methanol.

The extractive distillation temperature may be from about 50° C. to about 200° C., and the extractive distillation pressure may be from about 0 psia to about 20 psia. The extractive distillation temperature may be from about 50° C. to about 150° C. The extractive distillation temperature may be from about 80° C. to about 120° C. The extractive distillation temperature may be about 50° C., about 60° C., about 70° C., about 80° C., about 90° C., about 100° C., about 110° C., about 120° C., about 140° C., or about 150° C.

The extractive distillation pressure may be from about 0 psia to about 15 psia. The extractive distillation pressure may be from about 5 psia to about 10 psia. The extractive distillation pressure may be about 0 psia. The extractive distillation pressure may be about 1 psia. The extractive distillation pressure may be about 2 psia. The extractive distillation pressure may be about 3 psia. The extractive distillation pressure may be about 4 psia. The extractive distillation pressure may be about 5 psia. The extractive distillation pressure may be about 6 psia. The extractive distillation pressure may be about 7 psia. The extractive distillation pressure may be about 8 psia. The extractive distillation pressure may be about 9 psia. The extractive distillation pressure may be about 10 psia. The extractive distillation pressure may be about 11 psia. The extractive distillation pressure may be about 12 psia. The extractive distillation pressure may be about 13 psia. The extractive distillation pressure may be about 14 psia. The extractive distillation pressure may be about 15 psia.

The extractive distillation pressure may be from about 0 psig to about 20 psig. The extractive distillation pressure may be from about 0 psig to about 15 psig. The extractive distillation pressure may be about 0 psig. The extractive distillation pressure may be about 1 psig. The extractive distillation pressure may be about 2 psig. The extractive distillation pressure may be about 3 psig. The extractive distillation pressure may be about 4 psig. The extractive distillation pressure may be about 5 psig. The extractive distillation pressure may be about 9 psig. The extractive distillation pressure may be about 6 psig. The extractive distillation pressure may be about 7 psig. The extractive distillation pressure may be about 8 psig. The extractive distillation pressure may be about 9 psig. The extractive distillation pressure may be about 10 psig. The extractive distillation pressure may be about 11 psig. The extractive distillation pressure may be about 12 psig. The extractive distillation pressure may be about 13 psig. The extractive distillation pressure may be about 14 psig. The extractive distillation pressure may be about 15 psig.

The ethanol product mixture may comprise greater than about 90% (v/v) ethanol, greater than about 91% (v/v) ethanol, greater than about 92% (v/v) ethanol, greater than about 93% (v/v) ethanol, greater than about 94% (v/v) ethanol, greater than about 95% (v/v) ethanol, greater than about 96% (v/v) ethanol, greater than about 97% (v/v) ethanol, greater than about 98% (v/v) ethanol, or greater than about 99% (v/v) ethanol. The ethanol product mixture may comprise food-grade ethanol.

The ethanol product mixture may comprise between about 80% (v/v) to about 100% (v/v) ethanol. The ethanol product mixture may comprise between about 85% (v/v) to about 100% (v/v) ethanol. The ethanol product mixture may comprise between about 90% (v/v) to about 100% (v/v) ethanol. The ethanol product mixture may comprise between about 91% (v/v) to about 100% (v/v) ethanol. The ethanol product mixture may comprise between about 92% (v/v) to about 100% (v/v) ethanol. The ethanol product mixture may comprise between about 93% (v/v) to about 100% (v/v) ethanol. The ethanol product mixture may comprise between about 94% (v/v) to about 100% (v/v) ethanol. The ethanol product mixture may comprise between about 95% (v/v) to about 100% (v/v) ethanol. The ethanol product mixture may comprise between about 96% (v/v) to about 100% (v/v) ethanol. The ethanol product mixture may comprise between about 97% (v/v) to about 100% (v/v) ethanol. The ethanol product mixture may comprise between about 98% (v/v) to about 100% (v/v) ethanol. The ethanol product mixture may comprise between about 99% (v/v) to about 100% (v/v) ethanol.

The ethanol product mixture may comprise about 90% (v/v) ethanol. The ethanol product mixture may comprise about 91% (v/v) ethanol. The ethanol product mixture may comprise about 92% (v/v) ethanol. The ethanol product mixture may comprise about 93% (v/v) ethanol. The ethanol product mixture may comprise about 94% (v/v) ethanol. The ethanol product mixture may comprise about 95% (v/v) ethanol. The ethanol product mixture may comprise about 96% (v/v) ethanol. The ethanol product mixture may comprise about 97% (v/v) ethanol. The ethanol product mixture may comprise about 98% (v/v) ethanol. The ethanol product mixture may comprise about 99% (v/v) ethanol.

In certain embodiments, the ethanol product mixture consists essentially of ethanol. In further embodiments, the ethanol product mixture consists of ethanol.

In certain embodiments, the ethanol product mixture comprises propanol. The ethanol product mixture may comprise less than about 15% (v/v) propanol. The ethanol product mixture may comprise less than about 10% (v/v) propanol. The ethanol product mixture may comprise less than about 5% (v/v) propanol. The ethanol product mixture may comprise less than about 4% (v/v) propanol. The ethanol product mixture may comprise less than about 3% (v/v) propanol. The ethanol product mixture may comprise less than about 2% (v/v) propanol. The ethanol product mixture may comprise less than about 1% (v/v) propanol. The ethanol product mixture may comprise less than about 0.5% (v/v) propanol. The ethanol product mixture may comprise about 0.01% (v/v) to about 15% (v/v) propanol. The ethanol product mixture may comprise about 0.01% (v/v) to about 10% (v/v) propanol. The ethanol product mixture may comprise about 0.01% (v/v) to about 5% (v/v) propanol.

The ethanol product mixture may comprise about 0.01% (v/v) to about 1% (v/v) propanol. The ethanol product mixture may comprise about 10% (v/v) propanol, about 5% (v/v) propanol, about 4% (v/v) propanol, about 3% (v/v) propanol, about 2% (v/v) propanol, about 1% (v/v) propanol, or about 0.5% (v/v) propanol. In still further embodiments, the ethanol product mixture essentially does not comprise propanol.

In certain embodiments, the mass fraction of propanol to ethanol in the ethanol product mixture is lower than the mass fraction of ethanol to propanol in the alcohol feed by greater than about 70%. The mass fraction of propanol to ethanol in the ethanol product mixture may be lower than the mass fraction of ethanol to propanol in the alcohol feed by greater than about 75%. The mass fraction of propanol to ethanol in the ethanol product mixture may be lower than the mass fraction of ethanol to propanol in the alcohol feed by greater than about 80%. The mass fraction of propanol to ethanol in the ethanol product mixture may be lower than the mass fraction of ethanol to propanol in the alcohol feed by greater than about 85%. The mass fraction of propanol to ethanol in the ethanol product mixture may be lower than the mass fraction of ethanol to propanol in the alcohol feed by greater than about 90%. The mass fraction of propanol to ethanol in the ethanol product mixture may be lower than the mass fraction of ethanol to propanol in the alcohol feed by greater than about 91%. The mass fraction of propanol to ethanol in the ethanol product mixture may be lower than the mass fraction of ethanol to propanol in the alcohol feed by greater than about 92%. The mass fraction of propanol to ethanol in the ethanol product mixture may be lower than the mass fraction of ethanol to propanol in the alcohol feed by greater than about 93%. The mass fraction of propanol to ethanol in the ethanol product mixture may be lower than the mass fraction of ethanol to propanol in the alcohol feed by greater than about 94%. The mass fraction of propanol to ethanol in the ethanol product mixture may be lower than the mass fraction of ethanol to propanol in the alcohol feed by greater than about 95%. The mass fraction of propanol to ethanol in the ethanol product mixture may be lower than the mass fraction of ethanol to propanol in the alcohol feed by greater than about 96%. The mass fraction of propanol to ethanol in the ethanol product mixture may be lower than the mass fraction of ethanol to propanol in the alcohol feed by greater than about 97%. The mass fraction of propanol to ethanol in the ethanol product mixture may be lower than the mass fraction of ethanol to propanol in the alcohol feed by greater than about 98%. The mass fraction of propanol to ethanol in the ethanol product mixture may be lower than the mass fraction of ethanol to propanol in the alcohol feed by greater than about 99%.

In further embodiments, the mass fraction of propanol to ethanol in the ethanol product mixture is lower than the mass fraction of ethanol to propanol in the alcohol feed by about 70%, by about 75%, by about 80%, by about 85%, by about 90%, by about 91%, by about 92%, by about 93%, by about 94%, by about 95%, by about 96%, by about 97%, by about 98%, or by about 99%.

The alcohol feed may comprise methanol. The alcohol feed may comprise less than about 0.1% (v/v) methanol. The alcohol feed may comprise less than about 0.01% (v/v) methanol. The alcohol feed may comprise less than about 0.005% (v/v) methanol. The alcohol feed may comprise less than about 0.001% (v/v) methanol. The alcohol feed may comprise about 0.1% (v/v) methanol. The alcohol feed may comprise about 0.01% (v/v) methanol. The alcohol feed may comprise about 0.005% (v/v) methanol. The alcohol feed may comprise about 0.001% (v/v) methanol.

The propylene glycol mixture may comprise greater than about 80% (v/v) propylene glycol. The propylene glycol mixture may comprise greater than about 85% (v/v) propylene glycol. The propylene glycol mixture may comprise greater than about 90% (v/v) propylene glycol. The propylene glycol mixture may comprise greater than about 91% (v/v) propylene glycol. The propylene glycol mixture may comprise greater than about 92% (v/v) propylene glycol. The propylene glycol mixture may comprise greater than about 93% (v/v) propylene glycol. The propylene glycol mixture may comprise greater than about 94% (v/v) propylene glycol. The propylene glycol mixture may comprise greater than about 95% (v/v) propylene glycol. The propylene glycol mixture may comprise greater than about 96% (v/v) propylene glycol. The propylene glycol mixture may comprise greater than about 97% (v/v) propylene glycol. The propylene glycol mixture may comprise greater than about 98% (v/v) propylene glycol. The propylene glycol mixture may comprise greater than about 99% (v/v) propylene glycol.

The propylene glycol mixture may comprise between about 80% (v/v) propylene glycol to about 100% (v/v) propylene glycol. The propylene glycol mixture may comprise between about 85% (v/v) propylene glycol to about 100% (v/v) propylene glycol. The propylene glycol mixture may comprise between about 90% (v/v) propylene glycol to about 100% (v/v) propylene glycol. The propylene glycol mixture may comprise between about 91% (v/v) propylene glycol to about 100% (v/v) propylene glycol. The propylene glycol mixture may comprise between about 92% (v/v) propylene glycol to about 100% (v/v) propylene glycol. The propylene glycol mixture may comprise between about 93% (v/v) propylene glycol to about 100% (v/v) propylene glycol. The propylene glycol mixture may comprise between about 94% (v/v) propylene glycol to about 100% (v/v) propylene glycol. The propylene glycol mixture may comprise between about 95% (v/v) propylene glycol to about 100% (v/v) propylene glycol. The propylene glycol mixture may comprise between about 96% (v/v) propylene glycol to about 100% (v/v) propylene glycol. The propylene glycol mixture may comprise between about 97% (v/v) propylene glycol to about 100% (v/v) propylene glycol. The propylene glycol mixture may comprise between about 98% (v/v) propylene glycol to about 100% (v/v) propylene glycol. The propylene glycol mixture may comprise between about 99% (v/v) propylene glycol to about 100% (v/v) propylene glycol.

The propylene glycol mixture may comprise about 80% (v/v) propylene glycol. The propylene glycol mixture may comprise about 85% (v/v) propylene glycol. The propylene glycol mixture may comprise about 90% (v/v) propylene glycol. The propylene glycol mixture may comprise about 91% (v/v) propylene glycol. The propylene glycol mixture may comprise about 92% (v/v) propylene glycol. The propylene glycol mixture may comprise about 93% (v/v) propylene glycol. The propylene glycol mixture may comprise about 94% (v/v) propylene glycol. The propylene glycol mixture may comprise about 95% (v/v) propylene glycol. The propylene glycol mixture may comprise about 96% (v/v) propylene glycol. The propylene glycol mixture may comprise about 97% (v/v) propylene glycol. The propylene glycol mixture may comprise about 98% (v/v) propylene glycol. The propylene glycol mixture may comprise about 99% (v/v) propylene glycol.

In certain embodiments, the propylene glycol mixture further comprises ethanol. The propylene glycol mixture may comprise less than about 15% (v/v) ethanol. The propylene glycol mixture may comprise less than about 10% (v/v) ethanol. The propylene glycol mixture may comprise less than about 5% (v/v) ethanol. The propylene glycol mixture may comprise less than about 4% (v/v) ethanol. The propylene glycol mixture may comprise less than about 3% (v/v) ethanol. The propylene glycol mixture may comprise less than about 2% (v/v) ethanol. The propylene glycol mixture may comprise less than about 1% (v/v) ethanol. The propylene glycol mixture may comprise about 15% (v/v) ethanol. The propylene glycol mixture may comprise about 10% (v/v) ethanol. The propylene glycol mixture may comprise about 5% (v/v) ethanol. The propylene glycol mixture may comprise about 4% (v/v) ethanol. The propylene glycol mixture may comprise about 3% (v/v) ethanol. The propylene glycol mixture may comprise about 2% (v/v) ethanol. The propylene glycol mixture may comprise about 1% (v/v) ethanol.

In certain embodiments, the propylene glycol product mixture comprises greater than about 80% (v/v) propylene glycol. The propylene glycol product mixture may comprise greater than about 85% (v/v) propylene glycol. The propylene glycol product mixture may comprise greater than about 90% (v/v) propylene glycol. The propylene glycol product mixture may comprise greater than about 91% (v/v) propylene glycol. The propylene glycol product mixture may comprise greater than about 92% (v/v) propylene glycol. The propylene glycol product mixture may comprise greater than about 93% (v/v) propylene glycol. The propylene glycol product mixture may comprise greater than about 94% (v/v) propylene glycol. The propylene glycol product mixture may comprise greater than about 95% (v/v) propylene glycol. The propylene glycol product mixture may comprise greater than about 96% (v/v) propylene glycol. The propylene glycol product mixture may comprise greater than about 97% (v/v) propylene glycol. The propylene glycol product mixture may comprise greater than about 98% (v/v) propylene glycol. The propylene glycol product mixture may comprise greater than about 99% (v/v) propylene glycol. The propylene glycol product mixture may comprise about 80% (v/v) propylene glycol. The propylene glycol product mixture may comprise about 85% (v/v) propylene glycol. The propylene glycol product mixture may comprise about 90% (v/v) propylene glycol. The propylene glycol product mixture may comprise about 91% (v/v) propylene glycol. The propylene glycol product mixture may comprise about 92% (v/v) propylene glycol. The propylene glycol product mixture may comprise about 93% (v/v) propylene glycol. The propylene glycol product mixture may comprise about 94% (v/v) propylene glycol. The propylene glycol product mixture may comprise about 95% (v/v) propylene glycol. The propylene glycol product mixture may comprise about 96% (v/v) propylene glycol. The propylene glycol product mixture may comprise about 97% (v/v) propylene glycol. The propylene glycol product mixture may comprise about 98% (v/v) propylene glycol. The propylene glycol product mixture may comprise about 99% (v/v) propylene glycol. The propylene glycol product mixture may comprise between about 80% (v/v) propylene glycol to about 100% (v/v) propylene glycol. The propylene glycol product mixture may comprise between about 85% (v/v) propylene glycol to about 100% (v/v) propylene glycol. The propylene glycol product mixture may comprise between about 90% (v/v) propylene glycol to about 100% (v/v) propylene glycol. The propylene glycol product mixture may comprise between about 91% (v/v) propylene glycol to about 100% (v/v) propylene glycol. The propylene glycol product mixture may comprise between about 92% (v/v) propylene glycol to about 100% (v/v) propylene glycol. The propylene glycol product mixture may comprise between about 93% (v/v) propylene glycol to about 100% (v/v) propylene glycol. The propylene glycol product mixture may comprise between about 94% (v/v) propylene glycol to about 100% (v/v) propylene glycol. The propylene glycol product mixture may comprise between about 95% (v/v) propylene glycol to about 100% (v/v) propylene glycol. The propylene glycol product mixture may comprise between about 96% (v/v) propylene glycol to about 100% (v/v) propylene glycol. The propylene glycol product mixture may comprise between about 97% (v/v) propylene glycol to about 100% (v/v) propylene glycol. The propylene glycol product mixture may comprise between about 98% (v/v) propylene glycol to about 100% (v/v) propylene glycol. The propylene glycol product mixture may comprise between about 99% (v/v) propylene glycol to about 100% (v/v) propylene glycol. The propylene glycol product mixture may consist essentially of propylene glycol. The propylene glycol product mixture may consist of propylene glycol.

In certain embodiments, the propylene glycol product mixture comprises propanol. The propylene glycol product mixture may comprise less than about 15% (v/v) propanol, less than about 10% (v/v) propanol, less than about 5% (v/v) propanol, less than about 4% (v/v) propanol, less than about 3% (v/v) propanol, less than about 2% (v/v) propanol, or less than about 1% (v/v) propanol. The propylene glycol product mixture may comprise about 15% (v/v) propanol. The propylene glycol product mixture may comprise about 10% (v/v) propanol. The propylene glycol product mixture may comprise about 5% (v/v) propanol. The propylene glycol product mixture may comprise about 4% (v/v) propanol. The propylene glycol product mixture may comprise about 3% (v/v) propanol. The propylene glycol product mixture may comprise about 2% (v/v) propanol. The propylene glycol product mixture may comprise about 1% (v/v) propanol. The propylene glycol product mixture may comprise less than about 1% (v/v) propanol, less than about 0.5% (v/v) propanol, less than about 0.2% (v/v) propanol, less than about 0.1% (v/v) propanol, or less than about 0.05% (v/v) propanol. The propylene glycol product mixture may essentially include no propanol.

The propylene glycol product mixture may comprise greater than 80% propylene glycol and less than about 15% (v/v) propanol.

In certain embodiments, the propanol product mixture comprises greater than about 60% (v/v) propanol. The propanol product mixture may comprise greater than about 65% (v/v) propanol. The propanol product mixture may comprise greater than about 70% (v/v) propanol. The propanol product mixture may comprise greater than about 75% (v/v) propanol. The propanol product mixture may comprise greater than about 80% (v/v) propanol. The propanol product mixture may comprise greater than about 85% (v/v) propanol. The propanol product mixture may comprise greater than about 90% (v/v) propanol. The propanol product mixture may comprise greater than about 91% (v/v) propanol. The propanol product mixture may comprise greater than about 92% (v/v) propanol. The propanol product mixture may comprise greater than about 93% (v/v) propanol. The propanol product mixture may comprise greater than about 94% (v/v) propanol. The propanol product mixture may comprise greater than about 95% (v/v) propanol. The propanol product mixture may comprise greater than about 96% (v/v) propanol. The propanol product mixture may comprise greater than about 97% (v/v) propanol The propanol product mixture may comprise greater than about 98% (v/v) propanol. The propanol product mixture may comprise greater than about 99% (v/v) propanol. The propanol product mixture may comprise about 60% (v/v) propanol. The propanol product mixture may comprise about 65% (v/v) propanol. The propanol product mixture may comprise about 70% (v/v) propanol. The propanol product mixture may comprise about 75% (v/v) propanol. The propanol product mixture may comprise about 80% (v/v) propanol. The propanol product mixture may comprise about 85% (v/v) propanol. The propanol product mixture may comprise about 90% (v/v) propanol. The propanol product mixture may comprise about 91% (v/v) propanol. The propanol product mixture may comprise about 92% (v/v) propanol. The propanol product mixture may comprise about 93% (v/v) propanol. The propanol product mixture may comprise about 94% (v/v) propanol. The propanol product mixture may comprise about 95% (v/v) propanol. The propanol product mixture may comprise about 96% (v/v) propanol. The propanol product mixture may comprise about 97% (v/v) propanol The propanol product mixture may comprise about 98% (v/v) propanol. The propanol product mixture may comprise about 99% (v/v) propanol. The propanol product mixture may comprise between about 60% (v/v) propanol to about 100% (v/v) propanol. The propanol product mixture may comprise between about 65% (v/v) propanol to about 100% (v/v) propanol. The propanol product mixture may comprise between about 70% (v/v) propanol to about 100% (v/v) propanol. The propanol product mixture may comprise between about 75% (v/v) propanol to about 100% (v/v) propanol. The propanol product mixture may comprise between about 80% (v/v) propanol to about 100% (v/v) propanol. The propanol product mixture may comprise between about 85% (v/v) propanol to about 100% (v/v) propanol. The propanol product mixture may comprise between about 90% (v/v) propanol to about 100% (v/v) propanol. The propanol product mixture may comprise between about 91% (v/v) propanol to about 100% (v/v) propanol. The propanol product mixture may comprise between about 92% (v/v) propanol to about 100% (v/v) propanol. The propanol product mixture may comprise between about 93% (v/v) propanol to about 100% (v/v) propanol. The propanol product mixture may comprise between about 94% (v/v) propanol to about 100% (v/v) propanol. The propanol product mixture may comprise between about 95% (v/v) propanol to about 100% (v/v) propanol. The propanol product mixture may comprise between about 96% (v/v) propanol to about 100% (v/v) propanol. The propanol product mixture may comprise between about 97% (v/v) propanol to about 100% (v/v) propanol. The propanol product mixture may comprise between about 98% (v/v) propanol to about 100% (v/v) propanol. The propanol product mixture may comprise between about 99% (v/v) propanol to about 100% (v/v) propanol. In certain embodiments, the propanol product mixture consists essentially of propanol. In further embodiments, the propanol product mixture consists of propanol.

In certain embodiments, the propanol product mixture further comprises propylene glycol. The propanol product mixture may comprise less than about 15% (v/v) propylene glycol, less than about 10% (v/v) propylene glycol, less than about 5% (v/v) propylene glycol, less than about 4% (v/v) propylene glycol, less than about 3% (v/v) propylene glycol, less than about 2% (v/v) propylene glycol, or less than about 1% (v/v) propylene glycol. The propanol product mixture may comprise about 15% (v/v) propylene glycol. The propanol product mixture may comprise about 10% (v/v) propylene glycol. The propanol product mixture may comprise about 5% (v/v) propylene glycol. The propanol product mixture may comprise about 4% (v/v) propylene glycol. The propanol product mixture may comprise about 3% (v/v) propylene glycol. The propanol product mixture may comprise about 2% (v/v) propylene glycol. The propanol product mixture may comprise about 1% (v/v) propylene glycol. In further embodiments, the propanol product mixture may include no substantial amount of propylene glycol, e.g., less than about 1% (v/v) propylene glycol, less than about 0.5% (v/v) propylene glycol, or less than about 0.2% (v/v) propylene glycol.

The propanol product mixture further comprises ethanol. The propanol product mixture may comprise less than about 50% (v/v) ethanol. The propanol product mixture may comprise less than about 45% (v/v) ethanol. The propanol product mixture may comprise less than about 40% (v/v) ethanol. The propanol product mixture may comprise less than about 35% (v/v) ethanol. The propanol product mixture may comprise less than about 30% (v/v) ethanol. The propanol product mixture may comprise less than about 25% (v/v) ethanol. The propanol product mixture may comprise less than about 20% (v/v) ethanol. The propanol product mixture may comprise less than about 15% (v/v) ethanol. The propanol product mixture may comprise less than about 10% (v/v) ethanol. The propanol product mixture may comprise less than about 5% (v/v) ethanol. The propanol product mixture may comprise less than about 4% (v/v) ethanol. The propanol product mixture may comprise less than about 3% (v/v) ethanol. The propanol product mixture may comprise less than about 2% (v/v) ethanol. The propanol product mixture may comprise less than about 1% (v/v) ethanol. The propanol product mixture may comprise about 50% (v/v) ethanol. The propanol product mixture may comprise about 45% (v/v) ethanol. The propanol product mixture may comprise about 40% (v/v) ethanol. The propanol product mixture may comprise about 35% (v/v) ethanol. The propanol product mixture may comprise about 30% (v/v) ethanol. The propanol product mixture may comprise about 25% (v/v) ethanol. The propanol product mixture may comprise about 20% (v/v) ethanol. The propanol product mixture may comprise about 15% (v/v) ethanol. The propanol product mixture may comprise about 10% (v/v) ethanol. The propanol product mixture may comprise about 5% (v/v) ethanol. The propanol product mixture may comprise about 4% (v/v) ethanol. The propanol product mixture may comprise about 3% (v/v) ethanol. The propanol product mixture may comprise about 2% (v/v) ethanol. The propanol product mixture may comprise about 1% (v/v) ethanol.

In an embodiment, the propanol product mixture may include greater than 60% (v/v) propanol, less than 40% (v/v) ethanol, and less than about 10% (v/v) propylene glycol. The propanol product mixture may include greater than 70% (v/v) propanol, less than 30% (v/v) ethanol, and less than about 5% (v/v) propylene glycol. The propanol product mixture may include greater than 80% (v/v) propanol, less than 20% (v/v) ethanol, and less than about 5% (v/v) propylene glycol.

The step of separating the propylene glycol from the propanol in the propylene glycol mixture may be performed using a propylene glycol separator, which may be any distillation column known for use in the art in this way. Separating may be performed at a separation temperature and a separation pressure.

The separation temperature may be from about 50° C. to about 220° C., and the separation may be from about 0 psia to about 15 psia. The separation temperature may be from about 60° C. to about 210° C. The separation temperature may be from about 80° C. to about 200° C. The separation temperature may be about 60° C., about 70° C., about 80° C., about 90° C., about 100° C., about 110° C., about 120° C., about 130° C., about 140° C., about 150° C., about 160° C., about 170° C., about 180° C., about 190° C., about 200° C., or about 210° C.

The separation pressure may be from about 0 psia to about 15 psia. The separation pressure may be from about 5 psia to about 10 psia. The separation pressure may be from about 8 psia to about 12 psia. The separation pressure may be about 0 psia. The separation pressure may be about 1 psia. The separation pressure may be about 2 psia. The separation pressure may be about 3 psia. The separation pressure may be about 4 psia. The separation pressure may be about 5 psia. The separation pressure may be about 6 psia. The separation pressure may be about 7 psia. The separation pressure may be about 8 psia. The separation pressure may be about 9 psia. The separation pressure may be about 10 psia. The separation pressure may be about 11 psia. The separation pressure may be about 12 psia. The separation pressure may be about 13 psia. The separation pressure may be about 14 psia. The separation pressure may be about 15 psia.

In certain embodiments, all or a portion of the propylene glycol product mixture is combined with the propylene glycol feed such that the propylene glycol product mixture is a recycle stream. In certain embodiments, the propylene glycol product mixture is the propylene glycol feed, such that a separate stream of propylene glycol feed is not needed to meet the need of the system.

The method disclosed herein may further comprise a step of: distilling a crude alcohol feed comprising methanol, ethanol, and propanol at a crude alcohol distillation temperature and a crude alcohol distillation pressure to produce a methanol product mixture comprising methanol and the alcohol feed.

The crude alcohol distillation temperature may be from about 60° C. to about 150° C., and the crude alcohol distillation pressure may be from about 0 psia to about 20 psia. The crude alcohol distillation temperature may be from about 60° C. to about 100° C. The crude alcohol distillation temperature may be about 60° C. The crude alcohol distillation temperature may be about 70° C. The crude alcohol distillation temperature may be about 80° C. The crude alcohol distillation temperature may be about 90° C. The crude alcohol distillation temperature may be about 100° C.

The crude alcohol distillation pressure may be from about 0 psia to about 15 psia, about 0 psia to about 10 psia, or about 5 psia to about 15 psia. The crude alcohol distillation pressure may be about 0 psia. The crude alcohol distillation pressure may be about 1 psia. The crude alcohol distillation pressure may be about 2 psia. The crude alcohol distillation pressure may be about 3 psia. The crude alcohol distillation pressure may be about 4 psia. The crude alcohol distillation pressure may be about 5 psia. The crude alcohol distillation pressure may be about 6 psia. The crude alcohol distillation pressure may be about 7 psia. The crude alcohol distillation pressure may be about 8 psia. The crude alcohol distillation pressure may be about 9 psia. The crude alcohol distillation pressure may be about 10 psia. The crude alcohol distillation pressure may be about 11 psia. The crude alcohol distillation pressure may be about 12 psia. The crude alcohol distillation pressure may be about 13 psia. The crude alcohol distillation pressure may be about 14 psia. The crude alcohol distillation pressure may be about 15 psia.

The crude alcohol distillation pressure may be from about 0 psig to about 20 psig. The crude alcohol distillation pressure may be from about 0 psig to about 15 psig. The crude alcohol distillation pressure may be about 0 psig. The crude alcohol distillation pressure may be about 1 psig. The crude alcohol distillation pressure may be about 2 psig. The crude alcohol distillation pressure may be about 3 psig. The crude alcohol distillation pressure may be about 4 psig. The crude alcohol distillation pressure may be about 5 psig. The crude alcohol distillation pressure may be about 9 psig. The crude alcohol distillation pressure may be about 6 psig. The crude alcohol distillation pressure may be about 7 psig. The crude alcohol distillation pressure may be about 8 psig. The crude alcohol distillation pressure may be about 9 psig. The crude alcohol distillation pressure may be about 10 psig. The crude alcohol distillation pressure may be about 11 psig. The crude alcohol distillation pressure may be about 12 psig. The crude alcohol distillation pressure may be about 13 psig. The crude alcohol distillation pressure may be about 14 psig. The crude alcohol distillation pressure may be about 15 psig.

In certain embodiments, the mass fraction of methanol to ethanol in the alcohol feed is lower than the mass fraction of methanol to ethanol in the crude alcohol feed by greater than about 25%. The mass fraction of methanol to ethanol in the alcohol feed may be lower than the mass fraction of methanol to ethanol in the crude alcohol feed by greater than about 30%. The mass fraction of methanol to ethanol in the alcohol feed may be lower than the mass fraction of methanol to ethanol in the crude alcohol feed by greater than about 35%. The mass fraction of methanol to ethanol in the alcohol feed may be lower than the mass fraction of methanol to ethanol in the crude alcohol feed by greater than about 36%. The mass fraction of methanol to ethanol in the alcohol feed may be lower than the mass fraction of methanol to ethanol in the crude alcohol feed by greater than about 40%. The mass fraction of methanol to ethanol in the alcohol feed may be lower than the mass fraction of methanol to ethanol in the crude alcohol feed by greater than about 45%. The mass fraction of methanol to ethanol in the alcohol feed may be lower than the mass fraction of methanol to ethanol in the crude alcohol feed by greater than about 50%. The mass fraction of methanol to ethanol in the alcohol feed may be lower than the mass fraction of methanol to ethanol in the crude alcohol feed by greater than about 55%. The mass fraction of methanol to ethanol in the alcohol feed may be lower than the mass fraction of methanol to ethanol in the crude alcohol feed by greater than about 60%. The mass fraction of methanol to ethanol in the alcohol feed may be lower than the mass fraction of methanol to ethanol in the crude alcohol feed by greater than about 65%. The mass fraction of methanol to ethanol in the alcohol feed may be lower than the mass fraction of methanol to ethanol in the crude alcohol feed by greater than about 70%. The mass fraction of methanol to ethanol in the alcohol feed may be lower than the mass fraction of methanol to ethanol in the crude alcohol feed by greater than about 75%. The mass fraction of methanol to ethanol in the alcohol feed may be lower than the mass fraction of methanol to ethanol in the crude alcohol feed by greater than about 80%.

The mass fraction of methanol to ethanol in the alcohol feed may be lower than the mass fraction of methanol to ethanol in the crude alcohol feed by about 25%, by about 30%, by about 35%, by about 36%, by about 40%, by about 45%, by about 50%, by about 55%, by about 60%, by about 65%, by about 70%, by about 75%, or by about 80%.

In certain embodiments, the methanol product mixture essentially does not comprise ethanol. In further embodiments, the methanol product mixture essentially does not comprise ethanol or propanol.

In certain embodiments, the methanol product mixture consists essentially of methanol. In further embodiments, the methanol product mixture consists of methanol.

The method disclosed herein may further comprise a step of: separating a hydrated alcohol feed comprising water, methanol, ethanol, and propanol at a hydrated alcohol separation temperature and hydrated alcohol separation pressure to produce a waste water mixture comprising water and the crude alcohol feed.

The hydrated alcohol separation temperature may be from about 50° C. to about 200° C. The hydrated alcohol separation temperature may be from about 50° C. to about 150° C. The hydrated alcohol separation temperature may be about 50° C. The hydrated alcohol separation temperature may be about 60° C. In The hydrated alcohol separation temperature may be about 70° C. The hydrated alcohol separation temperature may be about 80° C. The hydrated alcohol separation temperature may be about 90° C. The hydrated alcohol separation temperature may be about 100° C. The hydrated alcohol separation temperature may be about 110° C. The hydrated alcohol separation temperature may be about 120° C. The hydrated alcohol separation temperature may be about 130° C. The hydrated alcohol separation temperature may be about 140° C. The hydrated alcohol separation temperature may be about 150° C.

The hydrated alcohol separation pressure may be from about 0 psia to about 20 psia. The hydrated alcohol separation pressure may be about 0 psia to about 15 psia, about 10 psia to about 15 psia, or about 0 psia to about 5 psia. The hydrated alcohol separation pressure may be about 0 psia. The hydrated alcohol separation pressure may be about 1 psia. The hydrated alcohol separation pressure may be about 2 psia. The hydrated alcohol separation pressure may be about 3 psia. The hydrated alcohol separation pressure may be about 4 psia. The hydrated alcohol separation pressure may be about 5 psia. The hydrated alcohol separation pressure may be about 6 psia. The hydrated alcohol separation pressure may be about 7 psia. The hydrated alcohol separation pressure may be about 8 psia. The hydrated alcohol separation pressure may be about 9 psia. The hydrated alcohol separation pressure may be about 10 psia. The hydrated alcohol separation pressure may be about 11 psia. The hydrated alcohol separation pressure may be about 12 psia. The hydrated alcohol separation pressure may be about 13 psia. The hydrated alcohol separation pressure may be about 14 psia. The hydrated alcohol separation pressure may be about 15 psia.

The hydrated alcohol separation pressure may be from about 0 psig to about 20 psig. The hydrated alcohol separation pressure may be from about 0 psig to about 15 psig. The hydrated alcohol separation pressure may be about 0 psig. The hydrated alcohol separation pressure may be about 1 psig. The hydrated alcohol separation pressure may be about 2 psig. The hydrated alcohol separation pressure may be about 3 psig. The hydrated alcohol separation pressure may be about 4 psig. The hydrated alcohol separation pressure may be about 5 psig. The hydrated alcohol separation pressure may be about 9 psig. The hydrated alcohol separation pressure may be about 6 psig. The hydrated alcohol separation pressure may be about 7 psig. The hydrated alcohol separation pressure may be about 8 psig. The hydrated alcohol separation pressure may be about 9 psig. The hydrated alcohol separation pressure may be about 10 psig. The hydrated alcohol separation pressure may be about 11 psig. The hydrated alcohol separation pressure may be about 12 psig. The hydrated alcohol separation pressure may be about 13 psig. The hydrated alcohol separation pressure may be about 14 psig. The hydrated alcohol separation pressure may be about 15 psig.

In certain embodiments, the hydrated alcohol feed comprises greater than about 50% (v/v) water. The hydrated alcohol feed may comprise greater than about 60% (v/v) water. The hydrated alcohol feed may comprise greater than about 70% (v/v) water. The hydrated alcohol feed may comprise greater than about 80% (v/v) water. The hydrated alcohol feed may comprise greater than about 90% (v/v) water. The hydrated alcohol feed may comprise greater than about 95% (v/v) water. The hydrated alcohol feed may comprise greater than about 99% (v/v) water. The hydrated alcohol feed may comprise about 50% (v/v) water. The hydrated alcohol feed may comprise about 60% (v/v) water. The hydrated alcohol feed may comprise about 70% (v/v) water. The hydrated alcohol feed may comprise about 80% (v/v) water. The hydrated alcohol feed may comprise about 90% (v/v) water. The hydrated alcohol feed may comprise about 95% (v/v) water. The hydrated alcohol feed may comprise about 99% (v/v) water. The hydrated alcohol feed may comprise between about 50% (v/v) water to about 100% (v/v) water. The hydrated alcohol feed may comprise between about 60% (v/v) water to less than about 100% (v/v) water. The hydrated alcohol feed may comprise between about 70% (v/v) water to about 99% (v/v) water. The hydrated alcohol feed may comprise between about 70% (v/v) water to about 90% (v/v) water. The hydrated alcohol feed may comprise between about 70% (v/v) water to about 85% (v/v) water. The hydrated alcohol feed may comprise between about 80% (v/v) water to about 99% (v/v) water.

In certain embodiments, the hydrated alcohol feed comprises less than about 50% (v/v) methanol. The hydrated alcohol feed may comprise less than about 45% (v/v) methanol. The hydrated alcohol feed may comprise less than about 40% (v/v) methanol. The hydrated alcohol feed may comprise less than about 35% (v/v) methanol. The hydrated alcohol feed may comprise less than about 30% (v/v) methanol. The hydrated alcohol feed may comprise about 10% (v/v) to about 50% (v/v) methanol, about 15% (v/v) to about 45% (v/v) methanol, about 20% (v/v) to about 40% (v/v) methanol, about 20% (v/v) to about 35% (v/v) methanol, or about 25% (v/v) to about 30% (v/v) methanol. The hydrated alcohol feed may comprise about 50% (v/v) methanol. The hydrated alcohol feed may comprise about 45% (v/v) methanol. The hydrated alcohol feed may comprise about 40% (v/v) methanol. The hydrated alcohol feed may comprise about 35% (v/v) methanol. The hydrated alcohol feed may comprise about 30% (v/v) methanol.

In certain embodiments, the hydrated alcohol feed comprises less than about 40% (v/v) ethanol, less than about 35% (v/v) ethanol, less than about 30% (v/v) ethanol, less than about 25% (v/v) ethanol, less than about 20% (v/v) ethanol, less than about 15% (v/v) ethanol, less than about 10% (v/v) ethanol, less than about 5% (v/v) ethanol, or less than about 1% (v/v) ethanol. The hydrated alcohol feed may comprise about 0.1% (v/v) to about 30% (v/v) ethanol, about 0.1% (v/v) to about 20% (v/v) ethanol, about 0.1% (v/v) to about 10% (v/v) ethanol, about 0.1% (v/v) to about 5% (v/v) ethanol, or about 0.1% (v/v) to about 1% (v/v) ethanol. The hydrated alcohol feed may comprise about 1% (v/v) ethanol. The hydrated alcohol feed may comprise about 0.5% (v/v) ethanol. The hydrated alcohol feed may comprise about 0.3% (v/v) ethanol.

The hydrated alcohol feed may comprise greater than about 50% (v/v) water, less than about 50% (v/v) methanol, and less than about 20% (v/v) ethanol. The hydrated alcohol feed may comprise greater than about 60% (v/v) water, less than about 40% (v/v) methanol, and less than about 20% (v/v) ethanol. The hydrated alcohol feed may comprise greater than about 70% (v/v) water, less than about 30% (v/v) methanol, and less than about 5% (v/v) ethanol. The hydrated alcohol feed may comprise about 60% (v/v) to less than about 100% (v/v) water, about 10% (v/v) to about 50% (v/v) methanol, and about 0.1% (v/v) to about 30% (v/v) ethanol.

In certain embodiments, the hydrated alcohol feed comprises propanol and/or butanol. The hydrated alcohol feed may comprise less than about 15% (v/v) propanol, less than about 10% (v/v) propanol, less than about 5% (v/v) propanol, or less than about 2% (v/v) propanol. The hydrated alcohol feed may comprise about 10% (v/v) to about 0.1% (v/v) propanol, about 5% (v/v) to about 0.1% (v/v) propanol, about 3% (v/v) to about 0.1% (v/v) propanol, or about 1% (v/v) to about 0.1% (v/v) propanol. The hydrated alcohol feed may comprise less than about 10% (v/v) butanol, less than about 5% (v/v) butanol, less than about 2% (v/v) butanol, or less than about 1% (v/v) butanol. The hydrated alcohol feed may comprise about 10% (v/v) to about 0.1% (v/v) butanol, about 5% (v/v) to about 0.1% (v/v) butanol, about 3% (v/v) to about 0.1% (v/v) butanol, or about 1% (v/v) to about 0.1% (v/v) butanol.

In certain embodiments, the crude alcohol feed further comprises water. The mass fraction of water to methanol/ethanol/propanol in the crude alcohol feed may be lower than the mass fraction of water to methanol/ethanol/propanol in the hydrated alcohol feed by greater than about 70%. The mass fraction of water to methanol/ethanol/propanol in the crude alcohol feed may be lower than the mass fraction of water to methanol/ethanol/propanol in the hydrated alcohol feed by greater than about 75%. The mass fraction of water to methanol/ethanol/propanol in the crude alcohol feed may be lower than the mass fraction of water to methanol/ethanol/propanol in the hydrated alcohol feed by greater than about 80%. The mass fraction of water to methanol/ethanol/propanol in the crude alcohol feed may be lower than the mass fraction of water to methanol/ethanol/propanol in the hydrated alcohol feed by greater than about 85%. The mass fraction of water to methanol/ethanol/propanol in the crude alcohol feed may be lower than the mass fraction of water to methanol/ethanol/propanol in the hydrated alcohol feed by greater than about 90%. The mass fraction of water to methanol/ethanol/propanol in the crude alcohol feed may be lower than the mass fraction of water to methanol/ethanol/propanol in the hydrated alcohol feed by about 70%. The mass fraction of water to methanol/ethanol/propanol in the crude alcohol feed may be lower than the mass fraction of water to methanol/ethanol/propanol in the hydrated alcohol feed by about 75%. The mass fraction of water to methanol/ethanol/propanol in the crude alcohol feed may be lower than the mass fraction of water to methanol/ethanol/propanol in the hydrated alcohol feed by about 80%. The mass fraction of water to methanol/ethanol/propanol in the crude alcohol feed may be lower than the mass fraction of water to methanol/ethanol/propanol in the hydrated alcohol feed by about 85%. The mass fraction of water to methanol/ethanol/propanol in the crude alcohol feed may be lower than the mass fraction of water to methanol/ethanol/propanol in the hydrated alcohol feed by about 90%.

The crude alcohol feed may comprise less than about 70% (v/v) water. The crude alcohol feed may comprise less than about 60% (v/v) water. The crude alcohol feed may comprise less than about 50% (v/v) water. The crude alcohol feed may comprise less than about 40% (v/v) water. The crude alcohol feed may comprise less than about 30% (v/v) water. The crude alcohol feed may comprise less than about 20% (v/v) water. The crude alcohol feed may comprise less than about 10% (v/v) water. The crude alcohol feed may comprise less than about 5% (v/v) water. The crude alcohol feed may comprise about 0.5% (v/v) to about 70% (v/v) water, about 1% (v/v) to about 60% (v/v) water, about 1% (v/v) to about 50% (v/v) water, about 1% (v/v) to about 40% (v/v) water, about 1% (v/v) to about 30% (v/v) water, about 1% (v/v) to about 20% (v/v) water, about 1% (v/v) to about 10% (v/v) water, or about 1% (v/v) to about 5% (v/v) water. The crude alcohol feed may comprise about 60% (v/v) water, about 50% (v/v) water, about 40% (v/v) water, about 30% (v/v) water, about 20% (v/v) water, about 10% (v/v) water, or about 5% (v/v) water.

In certain embodiments, the crude alcohol feed comprises greater than about 40% (v/v) methanol, greater than about 50% (v/v) methanol, greater than about 60% (v/v) methanol, greater than about 70% (v/v) methanol, greater than about 80% (v/v) methanol, greater than about 90% (v/v) methanol, or greater than about 95% (v/v) methanol. The crude alcohol feed may comprise about 45% (v/v) to about 99% (v/v) methanol, about 55% (v/v) to about 99% (v/v) methanol, about 60% (v/v) to about 99% (v/v) methanol, about 65% (v/v) to about 98% (v/v) methanol, about 70% (v/v) to about 99% (v/v) methanol about 75% (v/v) to about 99% (v/v) methanol, about 80% (v/v) to about 99% (v/v) methanol, about 85% (v/v) to about 99% (v/v) methanol, about 90% (v/v) to about 99% (v/v) methanol, or about 91% (v/v) to about 99% (v/v) methanol.

In certain embodiments, the crude alcohol feed comprises less than about 40% (v/v) ethanol, less than about 35% (v/v) ethanol, less than about 30% (v/v) ethanol, less than about 25% (v/v) ethanol, less than about 20% (v/v) ethanol, less than about 15% (v/v) ethanol, less than about 10% (v/v) ethanol, less than about 5% (v/v) ethanol, or less than about 2% (v/v) ethanol. The crude alcohol feed may comprise about 0.1% (v/v) to about 30% (v/v) ethanol, about 0.1% (v/v) to about 20% (v/v) ethanol, about 0.1% (v/v) to about 10% (v/v) ethanol, about 0.1% (v/v) to about 5% (v/v) ethanol, or about 0.5% (v/v) to about 2% (v/v) ethanol. The crude alcohol feed may comprise about 5% (v/v) ethanol. The crude alcohol feed may comprise about 0.5% (v/v) ethanol. The crude alcohol feed may comprise about 1% (v/v) ethanol.

The crude alcohol feed may comprise about 0.5% (v/v) to about 70% (v/v) water, about 45% (v/v) to about 99% (v/v) methanol, and about 0.1% (v/v) to about 30% (v/v) ethanol. The crude alcohol feed may comprise about 1% (v/v) to about 60% (v/v) water, about 55% (v/v) to about 99% (v/v) methanol, and about 0.1% (v/v) to about 20% (v/v) ethanol. The crude alcohol feed may comprise about 1% (v/v) to about 50% (v/v) water, about 65% (v/v) to about 99% (v/v) methanol, and about 0.1% (v/v) to about 10% (v/v) ethanol. The crude alcohol feed may comprise about 1% (v/v) to about 40% (v/v) water, about 75% (v/v) to about 99% (v/v) methanol, and about 0.1% (v/v) to about 5% (v/v) ethanol. The crude alcohol feed may comprise about 1% (v/v) to about 30% (v/v) water, about 0.5% (v/v) to about 2% (v/v) ethanol.

In certain embodiments, the crude alcohol feed further comprises propanol and/or butanol. The crude alcohol feed may comprise less than about 15% (v/v) propanol, less than about 10% (v/v) propanol, less than about 5% (v/v) propanol, less than about 2% (v/v) propanol or less than about 1% (v/v) propanol. The crude alcohol feed may comprise about 0.1% (v/v) to about 10% (v/v) propanol, about 0.1% (v/v) to about 5% (v/v) propanol, about 0.1% (v/v) to about 3% (v/v) propanol, or about 0.1% (v/v) to about 1% (v/v) propanol. The crude alcohol feed may comprise less than about 10% (v/v) butanol, less than about 5% (v/v) butanol, less than about 2% (v/v) butanol, or less than about 1% (v/v) butanol. The crude alcohol feed may comprise about 0.1% (v/v) to about 10% (v/v) butanol, about 0.1% (v/v) to about 5% (v/v) butanol, about 0.1% (v/v) to about 3% (v/v) butanol, or about 0.1% (v/v) to about 1% (v/v) butanol.

In certain embodiments, the crude alcohol feed comprises pentanol. The crude alcohol feed may comprise less than about 5% (v/v) pentanol, less than about 2% (v/v) pentanol, or less than about 1% (v/v) pentanol. The crude alcohol feed may comprise about 0.1% (v/v) to about 3% (v/v) pentanol, about 0.1% (v/v) to about 2% (v/v) pentanol, about 0.1% (v/v) to about 1% (v/v) propanol, or about 0.1% (v/v) to about 0.5% (v/v) pentanol. The crude alcohol feed may comprise about 0.1% (v/v) pentanol.

The waste water mixture may comprise greater than about 90% (v/v) water. The waste water mixture may comprise greater than about 91% (v/v) water. The waste water mixture may comprise greater than about 92% (v/v) water. The waste water mixture may comprise greater than about 93% (v/v) water. The waste water mixture may comprise greater than about 94% (v/v) water. The waste water mixture may comprise greater than about 95% (v/v) water. In yet further embodiments, the waste water mixture comprises greater than about 96% (v/v) water. The waste water mixture may comprise greater than about 97% (v/v) water. The waste water mixture may comprise greater than about 98% (v/v) water. The waste water mixture may comprise greater than about 99% (v/v) water. The waste water mixture may comprise about 90% (v/v) water, about 91% (v/v) water, about 92% (v/v) water, about 93% (v/v) water, about 94% (v/v) water, about 95% (v/v) water, about 96% (v/v) water, about 97% (v/v) water, about 98% (v/v) water, or about 99% (v/v) water.

The waste water mixture may comprise between about 90% (v/v) water to about 100% (v/v) water. The waste water mixture may comprise between about 91% (v/v) water to about 100% (v/v) water. The waste water mixture may comprise between about 92% (v/v) water to about 100% (v/v) water. The waste water mixture may comprise between about 93% (v/v) water to about 100% (v/v) water. The waste water mixture may comprise between about 94% (v/v) water to about 100% (v/v) water. The waste water mixture may comprise between about 95% (v/v) water to about 100% (v/v) water. The waste water mixture may comprise between about 96% (v/v) water to about 100% (v/v) water. The waste water mixture may comprise between about 97% (v/v) water to about 100% (v/v) water. The waste water mixture may comprise between about 98% (v/v) water to about 100% (v/v) water. The waste water mixture may comprise between about 99% (v/v) water to about 100% (v/v) water. In further embodiments, the waste water mixture consists essentially of water. In yet further embodiments, the waste water mixture consists of water.

In certain embodiments, the waste water mixture comprises methanol. The waste water mixture may comprise less than 10% (v/v) methanol. The waste water mixture may comprise less than 9% (v/v) methanol. The waste water mixture may comprise less than 8% (v/v) methanol. The waste water mixture may comprise less than about 7% (v/v) methanol. The waste water mixture may comprise less than about 6% (v/v) methanol. The waste water mixture may comprise less than about 5% (v/v) methanol. The waste water mixture may comprise less than about 4% (v/v) methanol. The waste water mixture may comprise less than about 3% (v/v) methanol. The waste water mixture may comprise less than about 2% (v/v) methanol. The waste water mixture may comprise less than about 1% (v/v) methanol. The waste water mixture may comprise about 8% (v/v) methanol, about 7% (v/v) methanol, about 6% (v/v) methanol, about 5% (v/v) methanol, about 4% (v/v) methanol, about 3% (v/v) methanol, about 2% (v/v) methanol, about 1% (v/v) methanol. The waste water mixture may comprise about 0.01% (v/v) to about 3% (v/v) methanol. The waste water mixture may comprise about 0.01% (v/v) to about 1% (v/v) methanol. The waste water mixture may comprise about 0.05% (v/v) to about 0.5% (v/v) methanol.

In certain embodiments, the waste water mixture consists of water and methanol. In certain embodiments, the waste water mixture consists of water and methanol, and is essentially free (e.g., less than about 0.01% (v/v), less than about 0.001% (v/v), or about 0.000% (v/v)) of propanol, ethanol, butanol, and/or pentanol.

### Systems for Extractive Distillation

Provided herein are systems for the purification of food-grade ethanol. A system disclosed herein comprises: an alcohol feed; a propylene glycol feed; an extractive separator; and a propylene glycol separator. The extractive separator may include an alcohol feed inlet, a propylene glycol feed inlet, an ethanol outlet, and a propylene glycol mixture outlet. The alcohol feed may be coupled to the alcohol feed inlet. The propylene glycol feed may be coupled to the propylene glycol feed inlet. The propylene glycol separator may comprise a propylene glycol mixture inlet, a propanol outlet, and a propylene glycol outlet. The propylene glycol mixture inlet may be coupled to the propylene glycol mixture outlet from the extractive separator.

In certain embodiments, the extractive separator is a first distillation column. In further embodiments, the first distillation column comprises metal packing. In yet further embodiments, the metal packing is random metal packing or structured metal packing.

In certain embodiments, the propylene glycol separator is a second distillation column. In further embodiments, the second distillation column comprises metal packing. In yet further embodiments, the metal packing is random metal packing or structured metal packing.

In certain embodiments, the propylene glycol outlet from the propylene glycol separator is coupled to the propylene glycol feed.

The system may further comprise: a crude alcohol feed; and a methanol separator comprising a crude alcohol feed inlet, a methanol outlet, and an alcohol feed outlet. The crude alcohol feed may be coupled to the crude alcohol feed inlet, and the alcohol feed outlet may be coupled to the alcohol feed.

In certain embodiments, the methanol separator is a third distillation column. In further embodiments, the third distillation column comprises metal packing. In yet further embodiments, the metal packing is random metal packing or structured metal packing.

The system may further comprise: a hydrated alcohol feed; and a water separator comprising a hydrated alcohol feed inlet, a water outlet, and a crude alcohol feed outlet. The hydrated alcohol feed may be coupled to the hydrated alcohol feed inlet, and the crude alcohol feed outlet may be coupled to the crude alcohol feed.

In certain embodiments, the water separator is a fourth distillation column. In further embodiments, the fourth distillation column comprises metal packing. In yet further embodiments, the metal packing is random metal packing or structured metal packing.

The system may further comprise: an ethanol purification column comprising an ethanol inlet and a pure ethanol outlet. The ethanol inlet may be coupled to the ethanol outlet and configured to receive the ethanol product mixture from the extractive separator. In certain embodiments, the ethanol purification column is a fifth distillation column. The ethanol purification column may be configured to remove impurities from the ethanol product mixture. The impurities may be water and other impurities. The impurities may be water, for example, in an amount of about 50 to about 200 ppm, or about 100 ppm. The fifth distillation column may comprise an absorbent bed. The absorbent bed may be made of any material known in the art to capture water and optionally other impurities from the ethanol product mixture. The absorbent bed may be packed with a molecular sieve. The impurities may be propylene glycol, for example, in an amount of about 1 to about 50 ppm, about 1 to about 10 ppm, or less than about 10 ppm. The fifth distillation column may separate ethanol from a trace amount of propylene glycol.

The system may include one or more heat exchangers, one or more condensers, and/or one or more reboilers placed downstream of each separator, as needed, as readily understood by one of ordinary skill in the art.

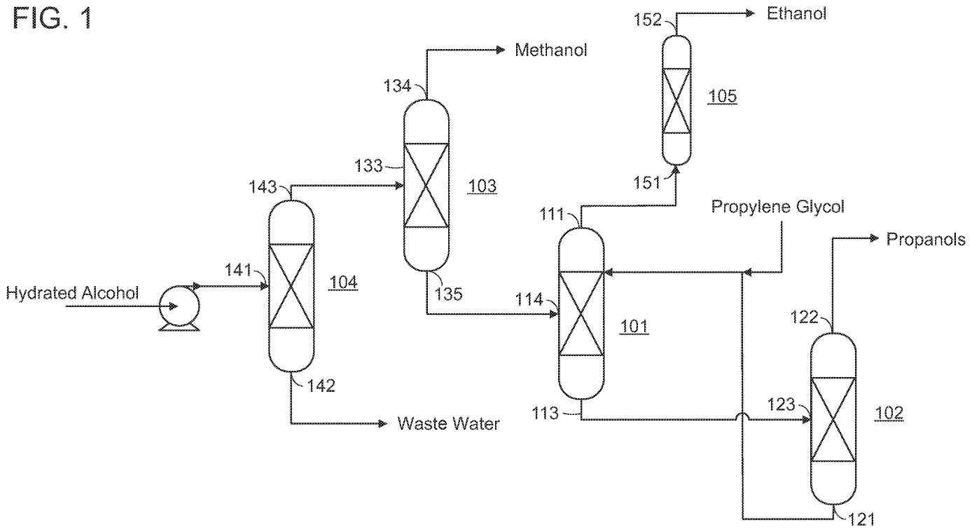

FIG. 1 is an example of a system for processing a hydrated alcohol feed into an ethanol product and also providing a propylene glycol product mixture that is a recycle stream. As shown in the figure, a hydrated alcohol feed is introduced into a water separator 104 having a hydrated alcohol feed inlet 141, a water outlet 142, and a crude alcohol feed outlet 143. The crude water outlet 143 is coupled to a crude water inlet 133 in a methanol separator 103. The methanol separator 103 also has a methanol outlet 134 and an alcohol feed outlet 135. The alcohol feed outlet 135 is coupled to an alcohol feed inlet 114 in an extractive separator 101. The extractive separator 101 also has a propylene glycol feed inlet 111, an ethanol outlet 112, and a propylene glycol mixture outlet 113. The ethanol outlet 112 is shown coupled to an ethanol outlet 151 in an ethanol purification column 105, which has a pure ethanol outlet 152. It will be readily envisioned that the system may exist and produce food-grade ethanol without the inclusion of this ethanol purification column. The propylene glycol mixture outlet 113 is coupled to a propylene glycol mixture inlet 123 in a propylene glycol separator 102. The propylene glycol separator 102 has a propanol outlet 122 and a propylene glycol outlet 121. The system is configured to combine the propylene glycol product mixture from the propylene glycol outlet 121 with the propylene glycol feed which is fed through the propylene glycol feed inlet 111. It will be readily envisioned that the system may exist without the combining of the propylene glycol product mixture from the propylene glycol outlet 121 with the propylene glycol feed.

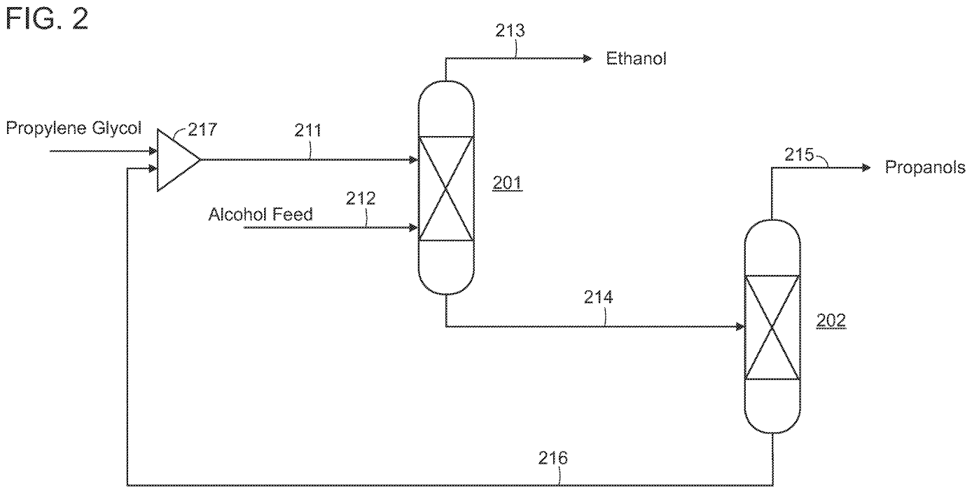

FIG. 2 is a system and method of the disclosure using two distillation columns for separating streams. As depicted therein, a propylene glycol feed 211 and an alcohol feed 212 are fed into an extractive distillation column 201 and combined (not shown) to form an ethanol product mixture 213 and a propylene glycol mixture 214. The propylene glycol mixture is provided to the propylene glycol separator 202 to form a propanol product mixture 215 and a propylene glycol product mixture 216. The propanol product mixture 215 may include a mixture of i-propanol and n-propanol. In this figure, the propylene glycol product mixture 216 is recycled and combined with the propylene glycol in a mixer 217 to make the propylene glycol feed 211, but it will be readily envisioned that the system may exist without the recycling the propylene glycol product mixture 216.

### Definitions

Unless otherwise defined herein, scientific and technical terms used in this application shall have the meanings that are commonly understood by those of ordinary skill in the art. Generally, nomenclature used in connection with, and techniques of, chemistry and chemical engineering described herein, are those well known and commonly used in the art.

The methods and techniques of the present disclosure are generally performed, unless otherwise indicated, according to conventional methods well known in the art and as described in various general and more specific references that are cited and discussed throughout this specification.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

In the present disclosure, certain components of these systems and methods involve “combining” reactants, feed streams, and the like, or involve reactants, feed streams, and the like which are described as being or having been “combined.” Reactants, feed streams, and the like may in some circumstances be combined before entry into a processing module (e.g., a reactor, reaction system, or separator), or may be individually fed into a processing module, such that they are combined within it. Thus, in some embodiments, these terms refer to the combination of feed streams, reactants, and the like prior to being fed into a processing module. In some embodiments, these terms describe the combination of feed streams, reactants, and the like within (or after being fed into) the subsequent reactor, reaction system, separator, etc.

In the present disclosure, certain components of these systems are described as being “coupled” to one another. As will be appreciated, the term “coupled” as used herein describes components that are operationally linked to one another, but does not preclude the presence of intervening components between those said to be coupled to one another. Additionally, as will be appreciated, various system components are described as “having” certain features. Such descriptions do not preclude, and specifically contemplate, the presence of additional features, such as inlets, outlets, valves, control mechanisms, measurement devices, heating and/or cooling systems, etc.

As used herein, the term “food-grade” means that the material, under conditions of good manufacturing practice, may be safely used as a food additive for human consumption or as a component of an article that contacts food for human consumption in accordance with the relevant regulatory (e.g., US FDA) requirements. In preferred embodiments, as will be understood by those of skill in the art, food-grade ethanol complies with US FDA standards. Specifically, food-grade ethanol meets the specifications of the “Food Chemicals Codex,” 4th ed. (1996), p. 136, which is expressly incorporated by reference herein in its entirety. In additional preferred embodiments, as will be understood by those of skill in the art, food-grade propylene glycol complies with US FDA standards. Specifically, food-grade propylene glycol meets the specifications of the Food Chemicals Codex, 3d Ed. (1981), p. 255, which is expressly incorporated by reference herein in its entirety.

As used herein, the term “molar ratio” refers to the ratio of equivalents in mole of X to equivalents in mole of Y, where X and Y can be, for example, different alcohols, water, or propylene glycol.

As used herein, the term “mass fraction” refers to the percentage of the mass of a substance in the total mass of the composition or a specific identified portion thereof.

### EXAMPLES

The invention now being generally described, it will be more readily understood by reference to the following examples which are included merely for purposes of illustration of certain aspects and embodiments of the present invention, and are not intended to limit the invention.

### Example 1: General Procedure for the Separation of Propanol from Ethanol

A feed mixture comprising 99.5% (v/v) ethanol and 0.5% (v/v) i-propanol was loaded into a flask containing 645 mL propylene glycol (1:2 molar ratio of feed mixture to propylene glycol) and twice distilled under one hour of reflux. Distillate samples were taken at 1.5 and 2 hours and analyzed via gas chromatography (Table 1). Additionally, the procedure was modeled in Aspen and the results are shown in Table 2.

TABLE 1
Experimental Data

| GC Sample | T_{bot} (C) | Tₜₒₚ (C) | vol % EtOH | vol % iPrOH | Vol % H₂O |
|---|---|---|---|---|---|
| PG 1.5 hr distillate | 114 | 76.2 | 86.11% | 0.029% | 13.861% |
| PG 2 hr distillate | 119 | 76.3 | 86.88% | 0.024% | 13.096% |

TABLE 2
Stream results for COL 1

| Mass Fractions | FEED | PG-FEED | ETHANOL | PG-IPA |
|---|---|---|---|---|
| Ethanol | 90.34% | 0.00% | 99.98% | 2.70% |
| i-Propanol | 0.25% | 0.00% | 0.02% | 0.13% |
| Propylene Glycol | 0.00% | 100.00% | 0.00% | 91.86% |
| n-Propanol | 9.41% | 0.00% | 0.00% | 5.31% |
| Mass Flows (lb/hr) | 31.17 | 50.73 | 26.67 | 55.23 |

### Example 2: General Procedure for the Recycling of Propylene Glycol

Propylene glycol recovery was carried out using a simple distillation. A feed comprising 1% (v/v) i-propanol, 85% (v/v) propylene glycol, and 14% (v/v) ethanol was twice distilled under one hour reflux in a column, and the recovered propylene glycol was then fed back into the propylene glycol feed. Additionally, the procedure was modeled in Aspen and the results are shown in Tables 3 and 4.

TABLE 3
Experimental Data for Extractant Recovery

| GC Sample | vol % iPrOH | vol % EtOH | vol % PG |
|---|---|---|---|
| Distillate | 1.18% | 98.80% | 0.02% |
| Bottoms | 0.00% | 0.04% | 99.96 |

TABLE 4
Stream results for COL 2

| Mass Fractions | PG-IPA | IPA-NPA | PG-RECYC |
|---|---|---|---|
| Ethanol | 2.70% | 33.15% | 0.00% |
| i-Propanol | 0.13% | 1.62% | 0.00% |
| Propylene Glycol | 91.86% | 0.02% | 100.00% |
| n-Propanol | 5.31% | 65.21% | 0.00% |
| Mass Flows (lb/hr) | 55.23 | 4.50 | 50.73 |

### Example 3: General Procedure for the Separation of Water from an Alcoholic Mixture

The separation of water from an alcoholic mixture was carried out using a distillation. A hydrated alcohol feed comprising 31.533% (v/v) methanol, 0.39% (v/v) ethanol, and 67.80% (v/v) water was separated both computationally (Table 5), and experimentally using Aspen (Table 6). Experimental conditions are as follows: Feed flow rate: 15 kg/hr, reflux rate: 9.25 kg/hr, bottom heating: 40% (v/v) or 35% (v/v). Alcohols were pushed to the top of the column, leaving water at the bottom of the column. After processing of stream 1, stream 2 is a crude alcohol feed and stream 3 is a waste water stream.

TABLE 5
Stream results for C-510

| Mass Fractions | 1 | 2 | 3 |
|---|---|---|---|
| Methanol | 26.86% | 95.27% | 0.10% |
| Ethanol | 0.33% | 1.19% | 0.00% |
| i-Propanol | 0.0030% | 0.011% | 0.00% |
| n-Propanol | 0.14% | 0.49% | 0.00% |
| n-butanol | 0.10% | 0.34% | 0.00% |
| n-Pentanol | 0.035% | 0.11% | 0.00% |
| Water | 72.53% | 2.58% | 99.89% |
| Mass Flow (kg/hr) | 15 | 4.22 | 10.78 |

TABLE 6
Water Distillation Experiment Results

| Sampling Position in | | | | | | |
|---|---|---|---|---|---|---|
| 4^{th} Distillation Column | MeOH | EtOH | iPrOH | 1-PrOH | 1-BuOH | 1-PtOH |
| (Heating Percentage) | Volume % | Volume % | Volume % | Volume % | Volume % | Volume % |
| Sample 1 Top Distillate (40%) | 94.28% | 0.75% | 0.019% | 0.35% | 0.22% | 0.11% |
| Sample 1 Bottom Distillate (40%) | 0.008% | 0 | 0 | 0 | 0 | 0 |
| Sample 2 Top Distillate (40%) | 97.05% | 0.71% | 0.018% | 0.26% | 0.12% | 0.051% |
| Sample 2 Bottom Distillate (40%) | 0.10% | 0 | 0 | 0 | 0 | 0 |
| Sample 3 Top Distillate (40%) | 99.25% | 0.73% | 0.018% | 0.22% | 0.061% | 0.018% |
| Sample 3 Bottom Distillate (40%) | 0.072% | 0 | 0 | 0 | 0 | 0 |
| Sample 4 Top Distillate (40%) | 96.87% | 0.82% | 0.019% | 0.43% | 0.32% | 0.17% |
| Sample 4 Bottom Distillate (40%) | 0.004% | 0 | 0 | 0 | 0 | 0 |
| Sample 5 Top Distillate (35%) | 92.33% | 0.69% | 0.017% | 0.27% | 0.15% | 0.068% |
| Sample 5 Bottom Distillate (35%) | 0.32% | 0 | 0 | 0 | 0 | 0 |
| Sample 6 Top Distillate (35%) | 96.74% | 0.82% | 0.020% | 0.43% | 0.29% | 0.13% |
| Sample 6 Bottom Distillate (35%) | 0.014% | 0 | 0 | 0 | 0 | 0 |
| Sample 7 Top Distillate (35%) | 96.82% | 0.73% | 0.018% | 0.27% | 0.19% | 0.044% |
| Sample 7 Bottom Distillate (35%) | 0.15% | 0 | 0 | 0 | 0 | 0 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method for the purification of food-grade ethanol comprising:
combining (a) an alcohol feed comprising ethanol and propanol with (b) a propylene glycol feed comprising propylene glycol to produce a purification feed;
distilling the purification feed at an extractive distillation temperature and an extractive distillation pressure to produce (a) an ethanol product mixture comprising ethanol and (b) a propylene glycol mixture comprising propylene glycol and propanol; and
separating the propylene glycol mixture at a separation temperature and a separation pressure to produce (a) a propanol product mixture comprising propanol and (b) a propylene glycol product mixture comprising propylene glycol.

2. (canceled)

3. The method of claim 1, wherein the extractive distillation temperature is about 50° C. to about 150° C.

4. The method of claim 1, wherein the extractive distillation pressure is about 0 psig to about 15 psig.

5. The method of claim 1, wherein the molar ratio of the alcohol feed to the propylene glycol feed is about 1:1 to about 1:3.

6. The method of claim 1, wherein the propanol is a mixture of n-propanol and i-propanol, and wherein the alcohol feed comprises less than about 10% (v/v) n-propanol.

7. The method of claim 6, wherein the alcohol feed comprises less than about 1% (v/v) i-propanol.

8. The method of claim 1, wherein the ethanol product mixture comprises between about 90% (v/v) ethanol to about 100% (v/v) ethanol.

9.-13. (canceled)

14. The method of claim 8, wherein the ethanol product mixture comprises less than about 10% (v/v) propanol; and wherein the mass fraction of propanol to ethanol in the ethanol product mixture is lower than the mass fraction of ethanol to propanol in the alcohol feed by greater than about 90%.

15.-16. (canceled)

17. The method of claim 1, wherein the propylene glycol mixture comprises ethanol in an amount of less than 10% (v/v) ethanol.

18. The method of claim 1, wherein propylene glycol product mixture comprises between about 90% (v/v) propylene glycol to about 100% (v/v) propylene glycol.

19. (canceled)

20. The method of claim 18, wherein the propylene glycol product mixture comprises propanol in an amount of less than about 10% (v/v) propanol.

21. The method of claim 1, wherein propanol product mixture comprises between about 60% (v/v) propanol to about 100% (v/v) propanol.

22. The method of claim 1, wherein the propanol product mixture further comprises propylene glycol.

23. The method of claim 22, wherein the propanol product mixture comprises less than about 10% (v/v) propylene glycol.

24. The method of claim 1, wherein the propanol product mixture further comprises ethanol.

25. The method of claim 24, wherein the propanol product mixture comprises less than about 40% (v/v) ethanol.

26. The method of claim 1, further comprising combining the propylene glycol product mixture with the propylene glycol feed.

27. The method of claim 1, further comprising:
distilling a crude alcohol feed comprising methanol, ethanol, and propanol at a crude alcohol distillation temperature and a crude alcohol distillation pressure to produce a methanol product mixture comprising methanol and the alcohol feed.

28.-32. (canceled)

33. The method of claim 27, wherein the methanol product mixture essentially does not comprise ethanol.

34. The method of claim 27, wherein the methanol product mixture essentially does not comprise ethanol or propanol.

35. The method of claim 27, further comprising:
separating a hydrated alcohol feed comprising water, methanol, ethanol, and propanol at a hydrated alcohol separation temperature and a hydrated alcohol separation pressure to produce a waste water mixture comprising water and the crude alcohol feed.

36.-46. (canceled)

47. A system for the purification of food-grade ethanol comprising:
an alcohol feed;
a propylene glycol feed;
an extractive separator comprising an alcohol feed inlet, a propylene glycol feed inlet, an ethanol outlet, and a propylene glycol mixture outlet; wherein the alcohol feed is coupled to the alcohol feed inlet, and the propylene glycol feed is coupled to the propylene glycol feed inlet; and
a propylene glycol separator comprising a propylene glycol mixture inlet, a propanol outlet, and a propylene glycol outlet; wherein the propylene glycol mixture inlet is coupled to the propylene glycol mixture outlet from the extractive separator.

48.-52. (canceled)

53. The system of claim 47, further comprising:
a crude alcohol feed; and
a methanol separator comprising a crude alcohol feed inlet, a methanol outlet, and an alcohol feed outlet; wherein the crude alcohol feed is coupled to the crude alcohol feed inlet, and the alcohol feed outlet is coupled to the alcohol feed.

54.-55. (canceled)

56. The system of claim 53, further comprising:
a hydrated alcohol feed; and
a water separator comprising a hydrated alcohol feed inlet, a water outlet, and a crude alcohol feed outlet; wherein the hydrated alcohol feed is coupled to the hydrated alcohol feed inlet, and the crude alcohol feed outlet is coupled to the crude alcohol feed.

57.-58. (canceled)
